(19) United States
(12) Patent Application Publication
(10) Pub. No.: US 2024/0337571 A1
(43) Pub. Date: Oct. 10, 2024

(54) Three-Point Bending Cylinder Asphalt Mixture Fatigue Test System

(71) Applicant: The Research Foundation for the State University of New York, Albany, NY (US)

(72) Inventor: Aksel Seitllari, Canton, NY (US)

(21) Appl. No.: 18/595,794

(22) Filed: Mar. 5, 2024

Related U.S. Application Data
(60) Provisional application No. 63/552,855, filed on Feb. 13, 2024.
(60) Provisional application No. 63/456,995, filed on Apr. 4, 2023.

(51) Int. Cl. G01N 3/20; G01N 3/06; G01N 33/42

(52) U.S. Cl. CPC G01N 3/20; G01N 3/06; G01N 33/42; G01N 2203/04

(57) ABSTRACT

A system and method for testing the durability of a cylindrical asphalt sample that is held within an apparatus and a load cell is moved by an actuator to apply a range of predetermined force against the cylindrical asphalt sample. The actuator is controlled by a computer device, further senses the resistance of the cylindrical asphalt sample to the predetermined force and communicates the sensed resistance data to the computer device. The computer device uses a Timoshenko Beam Theory (TBT) Model or a Viscoelastic Continuum Damage (VECD) Model to determine the deflection in the cylindrical asphalt sample to determine a durability measurement of the asphalt based upon the determined deflection.

### CROSS-REFERENCE TO RELATED APPLICATIONS

This application claims the benefit of U.S. Provisional Patent Application No. 63/456,995, filed Apr. 4, 2023, and U.S. Provisional Patent Application No. 63/552,855, filed Feb. 13, 2024, the entireties of which are hereby incorporated herein by this reference.

### BACKGROUND OF THE INVENTION

### 1. Field of the Invention

The present invention generally relates to systems and methods for testing the physical properties of materials. More particularly, the present invention relates to a three-point bending cylinder test particularly suited to test asphalt mixtures.

### 2. Description of the Related Art

Asphalt is the most commonly used material to pave roads and paths that physically support loads traveling thereover. The most common cause of deterioration of asphalt roads and paths is from “fatigue cracking.” Fatigue cracking is well-known to be one of the critical modes of distresses in asphalt pavements. Fatigue cracking phenomenon in asphaltic layers is caused by repeated traffic loading applications and predominantly happen in intermediate temperatures. Excessive exposure to tensile stress concentrations at the bottom and top of the asphalt layers eventually grow, coalesce, and lead to serious structural deterioration. Proper mix design, structural design, and enhanced material selection can significantly slow down the fatigue cracking and improve asphalt pavement service life. To better evaluate the resistance of asphalt pavements to fatigue cracking, numerous laboratory tests have been developed to mimic the traffic load applications in the field.

There are numerous physical testing mechanisms to determine the durability of asphalt to fatigue, such as bending tests and tension and compression tests. One particular testing device is a three-point bending flexural test which is well known as providing values for the modulus of elasticity in bending, including flexural stress, flexural strain, and the flexural stress-strain response of a material. This test is normally performed on a universal testing machine (tensile testing machine or tensile tester) with a three-point or four-point bend fixture. The main advantage of a three-point flexural test is the ease of the specimen preparation and testing. However, this method also has some disadvantages in that the results of the testing method are sensitive to specimen and loading geometry and strain rate. Therefore, these tests may be inaccurate to predict performance of a material in a real-world environment.

As an alternative to the bending flexural text, uniaxial push-pull and pull-pull tests have been gaining wide acceptance for fatigue evaluation of asphalt pavements because of their advantages over the flexural test. The advantages of those tests include homogenous stress-strain distribution throughout the sample geometry, the ability to produce samples using the Gyratory compactor, and straightforward application of the constitutive models to predict fatigue performance of asphalt pavements, such as the “Viscoelastic Continuum Damage” model.

However, one of the most challenging issues with the uniaxial testing is that two ends of the sample need to be cut parallel and the gluing end platens using a gluing jig can be cumbersome. As a result, many of the end-failures are experienced when sample ends are not cut in parallel fashion or gluing is not done properly. Since the samples are expected to fail in the center, many of the samples and their results are discarded, this leads to excessive sample preparation time and consumption of material. While the uniaxial testing is better than FPBB testing, it is currently not suitable as a routine testing alternative for balanced and performance-based mix design approaches.

Hence, there is a need for a test which addresses not only the above challenges but is also simple, sensitive to asphalt mix design, repeatable and practical. Current asphalt fatigue cracking tests are lengthy, cumbersome, and expensive to perform. Extensive material requirements are often needed for sample preparation with a large number of samples needed for testing. There are also common premature ‘end-failures’ leading to excessive sample preparation time and consumption of material. Furthermore, excessive equipment cost is common.

Thus, the prior art testing systems lack practical alternatives for balanced and performance-based mix design approaches. What is needed is a better asphalt testing system that is accurate, yet less expensive to operate and perform testing than the extant physical testing systems. It is thus to address this problem with current asphalt durability testing systems that the present invention is primarily directed.

### BRIEF SUMMARY OF THE INVENTION

Briefly described, the present invention includes a system and method testing the durability of an asphalt sample utilizing a three-point bending cylinder test of an asphalt sample. The system includes a sample-holding apparatus with a first clamp configured to selectively hold a first end of a cylindrical asphalt sample, a second clamp configured to hold a second end of a cylindrical asphalt sample, and a load cell that is selectively moveable at a range of predetermined force, the load cell configured to selectively apply to a predetermined force against a cylindrical asphalt sample held within the first clap and second clamp, the force applied on the cylindrical asphalt sample between the first end and second end of the cylindrical asphalt sample. The apparatus also has an actuator that selectively moves the load cell at a predetermined force, and the actuator further senses the resistance of the cylindrical asphalt sample to the predetermined force.

The system includes least one computer device communicatively connected to the actuator, and the computer device is configured to control the actuator to selectively move the load at a predetermined force against a cylindrical asphalt sample held in the first clamp and second clamp, receive sensed resistance data from the actuator from the predetermined force applied to the cylindrical asphalt sample, and determine a deflection of the cylindrical asphalt sample based upon a Timoshenko Beam Theory (TBT) Model or a Viscoelastic Continuum Damage (VECD) Model. The system can then determine a durability measurement of the cylindrical asphalt sample based upon the determined deflection and the appropriate model.

The computer device can be further configured to output the durability measurement of the cylindrical asphalt sample. Additionally, the sample-holding apparatus can further include a central clamp configured to selectively hold a cylindrical asphalt sample at a point in between the first end and second end of the cylindrical asphalt sample, with the cylindrical asphalt sample fixedly held within the first clamp, the second clamp, and the central clamp. In such configuration, the actuator can selectively moves the central clamp. Additionally, the cylindrical asphalt sample can be a core sample taken from an asphalt mixture and tested in accord with ASTM D8458-22.

In one embodiment, the computer device further be configured to treat the cylindrical asphalt sample as a linear elastic material within the TBT Model. Additionally, the computer device can be configured to base the VECD Model on an elastic-viscoelastic correspondence (E-VC) model of the cylindrical asphalt sample.

In one embodiment, the invention is intended to expand the capacities and be used as part of a new testing standard adopted for the Asphalt industry called ASTM D8458. The inventive system and method can vastly speed up, simplify, and reduce the cost of testing asphalt samples for stress and cracking under real-world simulations. To minimize the testing steps, the associated 3-point universal fixture is used to affix samples for standard testing tools for the asphalt industry, such as AMPTs (Asphalt Mixture Performance Tester) and/or MTS (Materials Testing System) machines. This improves the speed and accuracy of asphalt fatigue and cracking tests for fatigue characterization of asphalt mixes, either as part of a balanced mix design or mechanistic-empirical pavement structural design.

Several benefits of using the present inventive system and method are that they speed up and simplify testing, reduces the cost of testing, provides more accurate results, can be used with a variety of testing tools, improves the speed and accuracy of fatigue and cracking tests, and can be used for fatigue characterization of asphalt mixes. The system and method possesses the advantages of the current state of practice test, such as the uniaxial pull-pull fatigue test, but includes additional advantages such as not requiring a saw to cut the ends of the asphalt cylindrical sample, not requiring gluing operation and the gluing jig, and provides the possibility of estimating Poisson's ratio from the test data of the asphalt sample. Therefore, roadway agencies and those building roadways will have a better tool for assessing the susceptibility of asphalt mixtures to fatigue cracking.

The present system for asphalt testing system for durability could be used in a variety of applications, including research and development, quality control in asphalt productions, and evaluating the durability of existing pavements to identify areas that need to be repaired or replaced. Furthermore, the system and method could be used to educate and train engineers, technicians, and other professionals about asphalt durability.

The present invention therefore provides numerous advantages in more economically and accurately testing asphalt samples for their durability. Further, the present invention is industrially applicable in numerous actions surrounding the production and maintenance of asphalt for load-bearing surfaces, such as roadways, bridges, and pathways. Other advantages and features of the present invention would be apparent to one of skill in the art.

### BRIEF DESCRIPTION OF THE DRAWINGS

FIG. 1 is a perspective view of one embodiment of the apparatus for implementing the three-point bending cylinder test of an asphalt sample.

FIG. 2 is a side view of exaggerated deflection of the cylindrical asphalt sample in loading from the load cell.

FIG. 3A is a graph of the evolution of Poisson's ratio in a TBT model.

FIG. 3B is a graph of the evolution of |E*| value in a TBT model.

FIG. 4 is a table of a dynamic modulus master curve and shift factor coefficient for the mixtures in FIGS. 5A-5B.

FIG. 5A is a graph of linear viscoelastic properties of the mixtures in a log-log scale utilizing a VECD model.

FIG. 5B is a graph of linear viscoelastic properties of the mixtures in a semi-log scale plots of dynamic modulus master curves for 4E30SBS, 4E3SBS, and 4E3DVR utilizing a VECD model.

FIG. 6 is a flowchart of one embodiment of a method for testing the durability of a cylindrical asphalt sample.

### DETAILED DESCRIPTION OF THE INVENTION

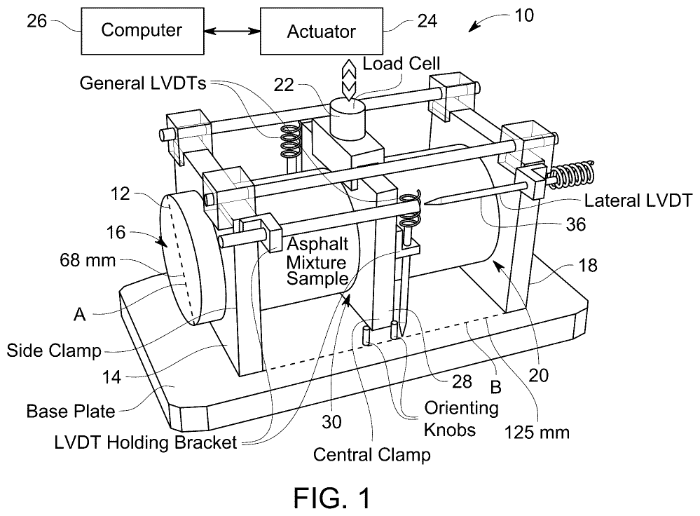

With reference to the figures in which like numerals represent like elements throughout the several views, FIG. 1 is a perspective view of one embodiment of the apparatus 10 of the system for implementing the three-point bending cylinder test of an asphalt sample 12. The sample-holding apparatus 10 has a first clamp 14 configured to selectively hold a first end 16 of a cylindrical asphalt sample 12, a second clamp 18 configured to hold a second end 20 of a cylindrical asphalt sample 12, and a load cell 22 that is selectively moveable at a range of predetermined force, the load cell 22 configured to selectively apply to a predetermined force against the cylindrical asphalt sample 12 held within the first clamp 14 and second clamp 18, the force applied on the cylindrical asphalt sample 12 between the first end 16 and second end 20 of the cylindrical asphalt sample 12. The apparatus 10 also has an actuator 24 that selectively moves the load cell 22 at a predetermined force, and the actuator 24 further senses the resistance of the cylindrical asphalt sample 12 to the predetermined force.

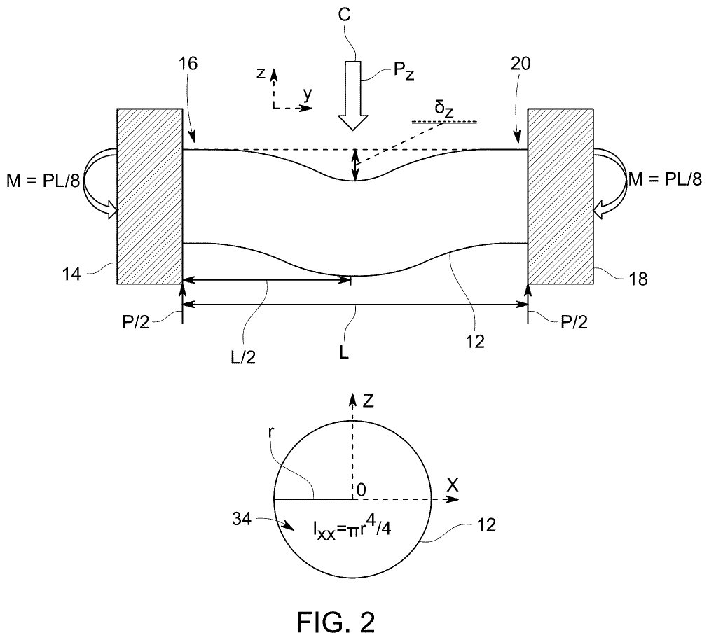

The system includes least one computer device 26 communicatively connected to the actuator 24, and the computer device 26 is configured to control the actuator 24 to selectively move the load at a predetermined force against a cylindrical asphalt sample 12 held in the first clamp 14 and second clamp 18, receive sensed resistance data from the actuator 24 from the predetermined force applied to the cylindrical asphalt sample 12, and determine a deflection of the cylindrical asphalt sample 12 based upon a Timoshenko Beam Theory (TBT) Model or a Viscoelastic Continuum Damage (VECD) Model, which are further described herein. The system can then determine a durability measurement of the cylindrical asphalt sample 12 based upon the determined deflection (FIG. 2) and the appropriate model.

The computer device 26 can be further configured to output the durability measurement of the cylindrical asphalt sample 12 as data to other computer devices or as a visual output such as from a display screen or monitor (not shown). Additionally, the sample-holding apparatus 10 can further include a central clamp 28 configured to selectively hold a cylindrical asphalt sample 12 at a central point 30 in between the first end 16 and second end 20 of the cylindrical asphalt sample, with the cylindrical asphalt sample 12 fixedly held within the first clamp 14, the second clamp 18, and the central clamp 28. In such configuration, the actuator 24 can selectively move the central clamp 28.

With specific regard to the testing of asphalt, ASTM D8458-22 provides a “Standard Test Method for Evaluation of Fatigue Performance of Asphalt Mixtures Using the Three-Point Bending Cylinder (3PBC) Test.” This test method is used to determine the fatigue resistance of asphalt mixtures normally at intermediate temperatures. The three-point bending cylinder test samples are obtained by coring a cylinder from the center of a gyratory compacted sample, or horizontal coring from field cores or slabs cut from field sections. After coring into a cylindrical shape, the asphalt sample is ready for testing. As embodied herein, the cylindrical asphalt sample 12 is a core sample taken from an asphalt mixture. Here, the dimensions of the cylindrical asphalt sample 12 are 68 mm in diameter (Line A) and greater than 125 mm in length (Line B).

The load cell 22 can be a servo-hydraulic or other device that selectively applies a mechanical force. The testing temperature of the cylindrical asphalt sample 12 can be altered to determine performance under various temperature conditions in accord with the weather. In this embodiment, a sinusoidal strain-controlled vertical load (with zero mean) is applied at the center point 30 of cylindrical asphalt sample 12 by the vertically free-moving central clamp 28 as illustrated in FIG. 1.

Here, the displacement measurements required for calculating the maximum strain at the bottom-center (or top-center) of the cylindrical asphalt sample 12 were obtained using two linear variable displacement transducers (LVDT) attached on both sides of the central clamp 28. The strain level was controlled through the actuator 24 and not the LVDTs. In addition, a third LVDT 36 can be attached to the top of the fixed supports to measure any potential lateral displacement due to steel bending, which is not desirable. Thus, the 3PBC is a zero-mean cyclic strain controlled test and the test stops when the microcracks propagate through the entire cylindrical asphalt sample diameter A (which can be observed visually). The stresses and strains for each cycle can be computed using the Timoshenko beam theory.

The Euler beam theory commonly used for the analysis of slender isotropic beams considers the beam kinematics in term of flexural stiffness. The low aspect ratio of 3PBC requires considerations of shear-induced deformations in the so-called “thickbeam”, i.e., Timoshenko beam theory. Analytical formulations for the stiffness of thick 3PBC are based on the following assumptions: The 3PBC cylindrical asphalt sample 12 is considered as a short beam with both ends clamped; The central clamp 28 restrains the cylindrical asphalt sample 12 from bending and moves parallel to first clamp 14 and second clamp 28; and Poisson's ratio of 3PBC is assumed to be constant during a particular test at certain frequency/temperature combination,

Initially, Ire Timoshenko beam theory has been presented herein by considering 3PBC as a linear elastic material. The formulations are then extended to viscoelastic behavior (for a given frequency/temperature combination) using the elastic-viscoelastic correspondence principle. For a linear elastic, isotropic-homogenous slender beam with both ends fixed and loaded at the center by a force P_{z}, the Euler theory states that the maximum vertical deflection (Oz) can be calculated as follows:

$${δ}_{z} = \frac{{P}_{z} {L}^{3}}{192 {EI}_{x x}}$$

where δ_{z} is the is the maximum vertical deflection, L is the span length, E is Young's modulus and Iₓₓ is the moment of inertia along axis xx. The schematic view of a fixed beam with a central load C is illustrated in FIG. 2

FIG. 2 is a side view of exaggerated deflection of the cylindrical asphalt sample 12 in loading from the load cell 22. FIG. 2 also includes a cross-section 34 of the cylindrical asphalt sample 12 further illustrating application of the Timoshenko beam theory. When the aspect ratio (length to diameter ratio) of a cylindrical beam is less than 6, the shear-induced deflection becomes significant and Euler theory does not apply.

For such beams, Timoshenko beam theory needs to be used to calculate the deflection as follows:

$${δ}_{z} = \frac{{P}_{z} {L}^{3}}{192 {EI}_{xx}} + \frac{2 β {P}_{z} L ( 1 + v )}{EA} \qquad [ 2 ]$$

where β is the shear coefficient, A is the cross-sectional area. Hutchinson derived the following expression for the shear coefficient (β) for beams with low aspect ratio:

$$β = \frac{6 {( 1 + v )}^{2}}{7 + 12 v + 4 {v}^{2}} \qquad [ 3 ]$$

where v is the Poisson's ratio. Equation [2] can be rearranged to yield elastic modulus as a function of the vertical load (P_{z}) and measured deflection at the center (δ_{z}):

$$E = \frac{{P}_{z} L [ {AL}^{2} + 384 β {I}_{xx} ( 1 + v ) ]}{192 {δ}_{z} {I}_{xx} A} \qquad [ 4 ]$$

For viscoelastic materials that are exposed to cyclic load at a constant frequency, Equation [4] can be used to calculate the magnitude of the dynamic modulus at each cycle N (i.e., |E*|N) as follows:

$${\vert {E}^{*} \vert}_{N} = K \frac{{P}_{z} ( N )}{{δ}_{z} ( N )} \qquad [ 5 ]$$

$$K = \frac{L [ {AL}^{2} + 384 β {I}_{xx} ( 1 + v ) ]}{192 {I}_{xx} A} \qquad [ 6 ]$$

where |E*|_{N} is the (damaged or undamaged) dynamic modulus at each cycle N, P_{z}(N) and δ_{z}(N) are the peak-to-peak load and deflection, respectively.

Selection of the appropriate Poisson's ratio is important for the accuracy of the |E*|_{N} in Equation [5]. It has been shown that the Poisson's ratio of an asphalt mixture is well correlated to the dynamic modulus (|E*|). Maher and Benner showed that the following relationship can be used to compute the Poisson's ratio from the |E*|:

$$v = a * ln \vert {E}^{*} \vert + b \qquad [ 7 ]$$

where a and b are the slope and intercept of the v−|E*| relationship.

One of the important advantages of the 3PBC test is that one can determine the a and b constants of v−|E*| relationship, i.e., the Poisson's ratio of an asphalt mixture. This can be achieved by running the 3PBC test at a relatively low load level (so that the sample is within linear viscoelastic range) at few temperatures/frequencies. Then the error between the |E*| computed using the Equations [5] through [7] (herein referred to as |E*| 3PBC) and corresponding |E*| obtained from the traditional uniaxial dynamic modulus test using the Asphalt Mixture Performance Tester (herein referred to as |E*|_{AMPT}) is minimized by varying the a and b constants (Equation [7]) as follows:

$${\vert {E}^{*} \vert}_{3 PBC} - {\vert {E}^{*} \vert}_{AMPT} = 0 \qquad [ 8 ]$$

The steps of estimating Poisson's ratio-|E*| relationship from 3PBC tests can be summarized as follows: 1) Run Dynamic Modulus (|E*|) tests in accordance with AASHTO T378 and generate the |E*| master curve in accordance with AASHTO R84; 2) Calculate the |E*| corresponding to the frequency and temperature combination for the planned 3PBC test. This |E*| is herein called |E*|_{AMPT}; 3) Run the 3PBC test at the planned frequency and temperature combination; 4) Assume initial a and b values for the Poisson's ratio formulation (Equation [7]); 5) Compute the Poisson's ratio using Equation [7], by using |E*| computed rom the dynamic modulus master curve obtained in step 1 (i.e. v=a*In|E*|_{AMPT}+b); 6) Plug in the Poisson's ratio computed in step 5 to Equation [6] to compute K. Then plug in the computed K (as well as P_{z} and δ_{z}) to Equation [5] to compute the |E*|. This |E*| is herein called |E*|_{3PBC}. 7) Compute the difference between |E*|_{3PBC} and |E*|_{AMPT}; 8) Vary the a and b values, repeat the steps 5, 6 and 7 until the error between |E*|_{3PBC} and |E*|_{AMPT} is minimized (Equation [8]).

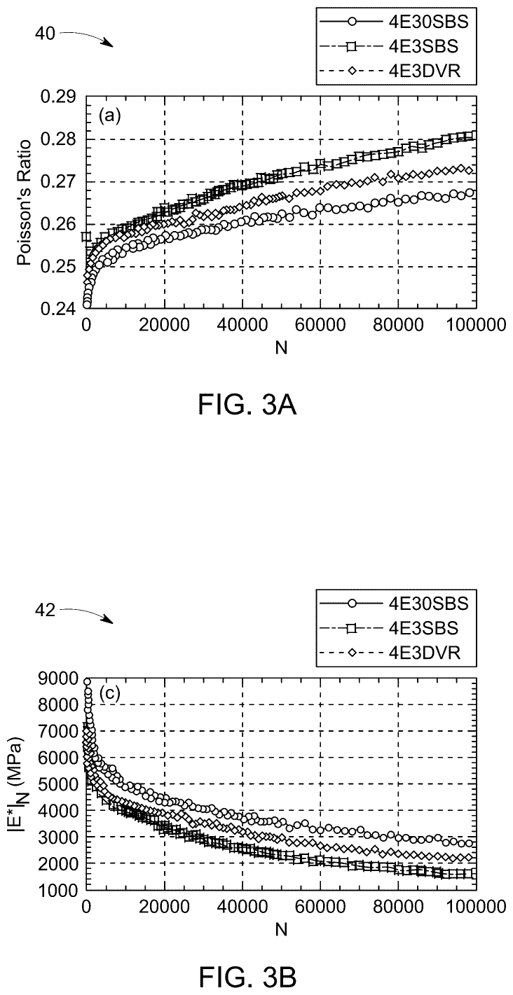

In order to verify this procedure to estimate a and b constants, a 3D Finite Element Analysis (FEA) was performed to simulate a perfect 3PBC test, using the exact geometry of the 3PBC sample and the fixtures. The results of the FEA analyses are shown in FIGS. 3A and 3B. In addition, actual 3PBC tests at a low strain (load) level were also conducted and the a and b constants computed and compared against the measured a and b constants reported in Maher and Benner! 2008b.

Once a and b constants (for the Poisson's ratio-|E*| relationship) are estimated for the linear viscoelastic (undamaged) state, they can be used in 3PBC fatigue tests in damaged state because Poisson's ratio is known not to change significantly during the fatigue tests. Since both left- and right-hand side of the Equation [5] includes the |E*|, another iterative procedure is needed. However, the fact that Poisson's ratio does not change significantly from one cycle to another can be used to simplify the computational steps.

A summary of the steps of computing |E*| at each cycle (i.e., |E*|_{N}) as damage grows are summarized as follows: 1) Using the known values of a and b constants, compute the Poisson's ratio using Equation [7] for cycle N=1 (initial condition), by using |E*| computed from the dynamic modulus master curve (i.e. (v_{N=I}=a*In|E*|_{AMPT}+b)). This condition is the undamaged state. 2) Since the Poisson's ratio does not change from one cycle to another significantly, it is assumed that v_{N+ΔN}≅vN; 3) Calculate β_{N+ΔN} using in Equation [3] and K_{N+ΔN} using Equation [6]. 4) For the next cycle N+ΔN, compute the damaged modulus, |E*|_{N+ΔN}, using Equation [5]. 5) For the next cycle N+ΔN, compute the Poisson's ratio using Equation [7], by using |E*| computed from the previous cycle (i.e. (v_{N=I}=a*In|E*|_{N}+b)); 6) Repeat the steps 2 through 5 for all subsequent cycles.

FIG. 3A is a graph 40 of the evolution of Poisson's ratio. FIG. 3B is a graph 42 of the evolution of |E*| values. FIGS. 3A-3B show examples of the evolution of (a) Poisson's ratio (v_{N}), and FIG. 3B, |E*|_{N} values with cycles during 3PBC tests. As shown in FIG. 3A, the Poisson's ratio increases with cycles (as expected), however, the total increase in v_{N} (at the end of 100,000 cycles) is about 10% of the initial value, which is relatively low. The increase in p is much lower than that of v_{N}, where the total increase at the end of 100,000 cycles is less than 1%. Therefore, these values may be assumed as constant to further simplify the formulations of 3PBC. Lastly, it should be noted that the maximum tensile and compressive stresses in the 3PBC sample occur on the farthest point from the neutral axis. For a circular cross-section the maximum tensile and compressive stresses are equal and can be obtained from the following expression:

$${( {σ}_{y} )}_{max} = - {( {σ}_{y} )}_{min} = \frac{M}{S} \qquad [ 9 ]$$

where, σ_{y} is the normal stress, M is the maximum bending moment at the center of the beam (M=P_{Z}L/8 for the 3PBC setup), S is the section modulus, which (for a solid circular cross-section) is calculated as S=πα^{β}/32 (where d=diameter). Even though Equation [9] was developed for pure bending with no shear presence, studies have shown that the effect of shear on normal stresses is negligible (American Wood Council 2005; Timoshenko and Gere 1972).

Alternately, the Viscoelastic Continuum Damage VECD theory can be used for fatigue characterization of asphalt mixtures significantly to reduce the experimental burden required to calibrate phenomenological fatigue life formulation (i.e., N_{f}=αε^{-b} E^{-c}. Extensive literature exists on the VECD and its practical applications in characterizing uniaxial fatigue behavior of asphalt mixtures. There are also several recent studies that have presented the application of the VECD theory on the analysis of fatigue behavior on flexural tests. The VECD constitutive model is based on Schapery's proposed elastic-viscoelastic correspondence (E-VC) principle, which can be applied to both linear and non-linear viscoelastic materials, and work potential theory (Schapery 1990). The E-VC principle states that the constitutive equations for a particular viscoelastic media are equivalent to equations of elastic media when the concept of pseudostrain is used in lieu of actual physical strain. The pseudostrain in time domain can be computed using the following convolution integral:

$${ε}^{R} = \frac{1}{{E}^{R}} {\int }_{0}^{t} E ( t - τ ) \frac{d ε}{d τ} d τ$$

where ε^{R} is the pseudostrain, E_{R} is a reference modulus often set as unity. Once E_{R} is assumed as unity, ε^{R} corresponds to linear viscoelastic stress, E(t) is the linear viscoelastic relaxation modulus, t is the time and τ is the time variable of integration.

The amount of work required for initiation and coalescing of microcracks is conveniently determined by the use of damage parameters (internal state variables). The mathematical approach to describe these phenomena includes the simplest form of pseudostrain energy density function and a single internal state variable S described as follows (Park, Kim, and Schapery 1996):

$$σ = \frac{\partial {W}^{R}}{\partial {ε}^{R}} = C ( S ) {ε}^{R}$$

$${W}^{R} = \frac{I}{2} C ( S ) {ε}^{{R}^{2}}$$

$$\frac{dS}{dt} = {( - \frac{\partial {W}^{R}}{\partial S} )}^{α}$$

where σ is the stress, W^{R} is the pseudostrain energy density function, C(S) is pseudo-stiffness as a function of a single damage parameter, dS/dt represents the damage evolution rate, I is an initial stiffness parameter used to eliminate the sample to sample variability, tis time and α is a constant related to the rate of damage growth in viscoelastic media (α=1/m, where m is the maximum slope of the relaxation modulus master curve in log-log scale).

In the case of cyclic loading at a constant frequency with no rest periods which is also applied in this study, the pseudostrain (ε^{R}) and the pseudostiffness (C) values at each cycle can be calculated as follows:

$${C}_{N} = \frac{{\vert {E}^{*} \vert}_{N}}{{\vert {E}^{*} \vert}_{LVE}}$$

$${ε}_{N}^{R} = \frac{{σ}_{N}}{{C}_{N}}$$

where, |E*|_{LVE} is the linear viscoelastic dynamic modulus (i.e., |E*|_{LVE=}|E*|_{N=1}), |E*|_{N} is the dynamic modulus and ε₀^{N} is the peak strain measured at the Nth cycle.

The damage parameter(S) at the peak of each cycle was calculated using a practical procedure proposed by Kutay et al. (Kutay, Gibson, and Youtcheff 2008):

$${S}_{N + Δ N} = {S}_{N} + {{( \frac{Δ N}{{f}_{R}} )}^{\frac{1}{1 + α}} [ - 0.5 I {ε}_{N}^{{R}^{2}} ( {C}_{N + Δ N} - {C}_{N} ) ]}^{\frac{α}{1 + α}}$$

where ΔN is the cycle increment and f_{R} is the reduced frequency.

It should be noted that the damage characteristic curve (C-S) is a unique curve that can be used to predict the fatigue life of asphalt mixtures at different frequencies and temperatures at a required strain level. The C-S curve is computed from the peak-to-peak stress-strain data retrieved for each cycle. However, the C-S curve should not be used to rank the fatigue performance of different mixtures since it is a normalized curve. Mixture classification should be done based on their fatigue life (number of cycles to failure (N_{f})) at a certain temperature, frequency and strain level.

It is important to select a fatigue failure criterion, which defines the Nt. Numerous failure criteria have been investigated by different researchers. Different failure criteria existing in the literature and compared the fatigue failure results against the field cracking data retrieved from the FHWA's accelerated pavement testing facility (APT). Selected failure criteria have the same trends with the FHWA's APT field cracking data. Hence, a 50% reduction in stiffness was recommended, therefore applied in the present invention. The fatigue life (N_{f}) was calculated using the following expression derived for the specific case of cyclic tests at constant frequency with no rest periods (Kutay et al. 2009):

$${N}_{f} = {\sum }_{S = 1}^{{S}_{f}} {[ - \frac{{ε}_{0}^{2} {\vert {E}^{*} \vert}_{LVE}^{2}}{2} \frac{dC}{dS} {\vert}_{at S} ]}^{- α} f Δ {S}_{S}$$

Where Sf is the damage parameter value corresponding to C=0.5.

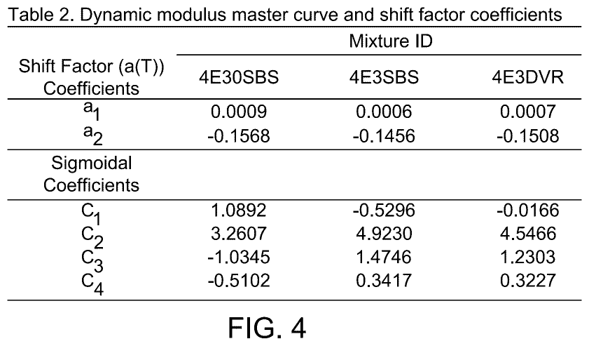

One of the prerequisite steps of the VECD-based characterization is the determination of the linear viscoelastic |E*| master curve. Therefore, |E*| test needs to be run for analysis of both 3PBC and PP test results. Here, the following shift factor and sigmoidal relationships were used to construct the |E*| master curve:

$$log ( {a}_{T} ( T ) ) = {a}_{1} ( {T}^{2} - {T}_{ref}^{2} ) + {a}_{2} ( T - {T}_{ref} )$$

$$log ( \vert {E}^{*} \vert ) = {c}_{1} + \frac{{c}_{2}}{1 + {e}^{( - {c}_{3} - {c}_{4} log ( {f}_{R} ) )}}$$

where T_{ref} is the reference temperature, a₁ and a₂ are the shift factors polynomial coefficients, c₁, c₂, c₃, c₄ are the sigrnoidal coefficients, f_{R} is the reduced frequency (f_{R}=fa(T)). T_{ref} was selected as 20° C. The dynamic modulus master curve and shift factor coefficients are shown in the table of FIG. 4.

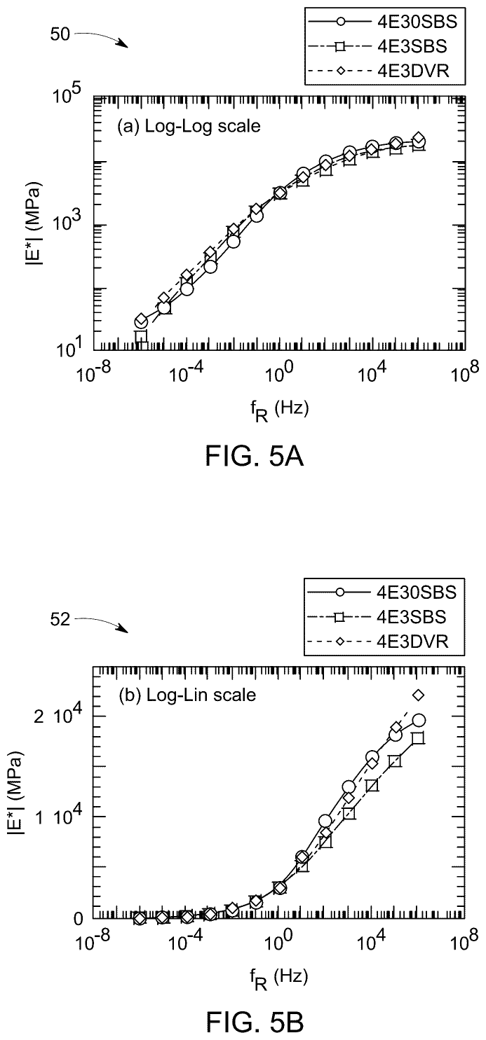

FIG. 5A is a graph 50 of linear viscoelastic properties of the mixtures in a log-log scale utilizing a VECD model. FIG. 5B is a graph 52 of linear viscoelastic properties of the mixtures in a semi-log scale plots of dynamic modulus master curves for 4E30SBS, 4E3SBS, and 4E3DVR utilizing a VECD model. FIGS. 5A-5B illustrate the |E*| master curves in log scale and semi-log scale for each mixture, respectively. Overall, all mixtures had similar |E*| values. The 4E3DVR was slightly stiffer than the two SBS modified mixtures.

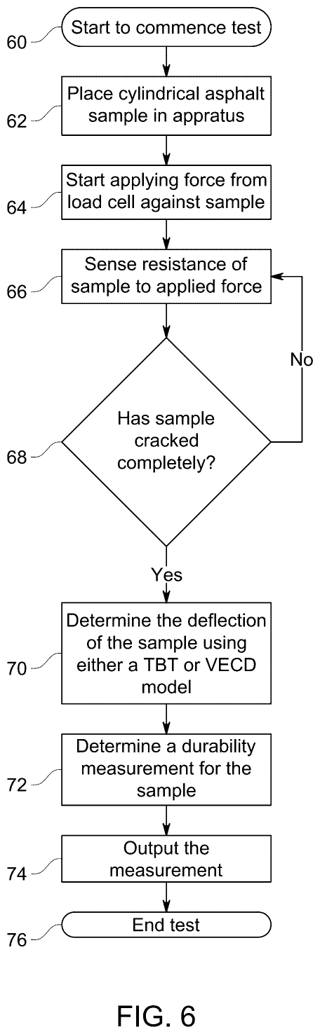

FIG. 6 is a flowchart of one embodiment of a method for testing the durability of a cylindrical asphalt sample 12. The method for testing the durability of an asphalt sample starts, as shown Start 60, with placing a cylindrical asphalt sample 12 in a sample-holding apparatus 10, as shown at step 62, the cylindrical asphalt sample 12 having a first end 16 and second end 20 thereof, the sample-holding apparatus 10 including a first clamp 14 configured to selectively hold the first end 16 of a cylindrical asphalt sample 12, and second clamp 18 configured to hold the second end 20 of the cylindrical asphalt sample 12, and then selectively applying a predetermined force, as shown at step 64, against the cylindrical asphalt sample 12 held within the first clamp and second clamp 18 a load cell 22 selectively moveable at a range of predetermined force, the predetermined force applied from load cell 22 configured to apply force on the cylindrical asphalt sample 12 at a point 30 between the first end 16 and second end 20 of the cylindrical asphalt sample 12 via an actuator 24 selectively moving the load cell 22.

The method continues by sensing at the actuator 24, as shown at step 66, the resistance of the cylindrical asphalt sample 12 to the predetermined force, and then receiving the sensed resistance data from the actuator 24 from the predetermined force applied to the cylindrical asphalt sample 12 and determining, as shown at decision 68, if the sample has failed (here, cracking completely through the cylindrical asphalt sample 12) as shown at decision 68. The step of receiving can be at the computer device 26. Further, the method includes determining, as shown at step 70, a deflection of the cylindrical asphalt sample 12 based upon one of a Timoshenko Beam Theory (TBT) Model (described above), or a Viscoelastic Continuum Damage (VECD) Model (described above), and then determining a durability measurement of the cylindrical asphalt sample 12 based upon the determined deflection, as shown at step 72.

The method can further include further outputting the durability measurement of the cylindrical asphalt sample 12, as shown at step 74, which can occur from the computer 26. The outputting could be sending the data or simply visually displaying the durability measurement. After outputting the measurement, the method of testing can then conclude as shown at end 76.

When the sample-holding apparatus 10 further includes a central clamp 28 configured to selectively hold a cylindrical asphalt sample 12 at a point 30 in between the first end 16 and second end 18 of the cylindrical asphalt sample 12, the method can further includes selectively moving the central clamp. The method can also further treating the cylindrical asphalt sample 12 as a linear elastic material within a TBT Model, as described above. The method can also include basing the VECD Model on an elastic-viscoelastic correspondence (E-VC) model of the cylindrical asphalt sample, as is further described above. Further, the method can include a physical step of creating the cylindrical asphalt sample 12 by taking a core sample from an asphalt mixture in the appropriate dimensions to fit within the sample-holding apparatus 10.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments herein were chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for testing a durability of an asphalt sample, comprising:
a sample-holding apparatus, including:
a first clamp configured to selectively hold a first end of a cylindrical asphalt sample;
a second clamp configured to hold a second end of a cylindrical asphalt sample;
a load cell selectively moveable at a range of predetermined force, the load cell configured to selectively apply to a predetermined force against a cylindrical asphalt sample held within the first clamp and second clamp, the force applied on the cylindrical asphalt sample between the first end and second end of the cylindrical asphalt sample; and
an actuator selectively moving the load cell at a predetermined force, the actuator further sensing a resistance of the cylindrical asphalt sample to the predetermined force; and
at least one computer device communicatively connected to the actuator, wherein the at least one computer device configured to:
control the actuator to selectively move the load at a predetermined force against a cylindrical asphalt sample held in the first clamp and second clamp;
receive sensed resistance data from the actuator from the predetermined force applied to the cylindrical asphalt sample;
determine a deflection of the cylindrical asphalt sample based upon a Timoshenko Beam Theory (TBT) Model; and
determine a durability measurement of the cylindrical asphalt sample based upon the determined deflection.

2. The system of claim 1, wherein at least one computer device further configured to output the durability measurement of the cylindrical asphalt sample.

3. The system of claim 1, wherein the sample-holding apparatus further includes a central clamp configured to selectively hold a cylindrical asphalt sample at a point in between the first end and second end of the cylindrical asphalt sample.

4. The system of claim 3, further comprising a cylindrical asphalt sample fixedly held within the first clamp, the second clamp, and the central clamp.

5. The system of claim 3, wherein the actuator selectively moves the central clamp.

6. The system of claim 1, wherein the at least one computer device further configured to treat the cylindrical asphalt sample as a linear elastic material within the TBT Model.

7. The system of claim 4, wherein the cylindrical asphalt sample is a core sample taken from an asphalt mixture.

8. A system for testing a durability of an asphalt sample, comprising:
a sample-holding apparatus, including:
a first clamp configured to selectively hold a first end of a cylindrical asphalt sample;
a second clamp configured to hold a second end of a cylindrical asphalt sample;
a load cell selectively moveable at a range of predetermined force, the load cell configured to selectively apply to a predetermined force against cylindrical asphalt sample held within the first clamp and second clamp, the force applied on the cylindrical asphalt sample between the first end and second end of the cylindrical asphalt sample; and
an actuator selectively moving the load cell at a predetermined force, the actuator further sensing a resistance of the cylindrical asphalt sample to the predetermined force; and
at least one computer device communicatively connected to the actuator, wherein the at least one computer device configured to:
control the actuator to selectively move the load at a predetermined force against a cylindrical asphalt sample held in the first clamp and second clamp;
receive sensed resistance data from the actuator from the predetermined force applied to the cylindrical asphalt sample;
determine a deflection of the cylindrical asphalt sample based upon a Viscoelastic Continuum Damage (VECD) Model; and
determine a durability measurement of the cylindrical asphalt sample based upon the determined deflection.

9. The system of claim 8, wherein at least one computer device further configured to output the durability measurement of the cylindrical asphalt sample.

10. The system of claim 8, wherein the sample-holding apparatus further includes a central clamp configured to selectively hold a cylindrical asphalt sample at a point in between the first end and second end of the cylindrical asphalt sample.

11. The system of claim 10, further comprising a cylindrical asphalt sample fixedly held within the first clamp, the second clamp, and the central clamp.

12. The system of claim 10, wherein the central clamp attached to the actuator, and the actuator selectively moves the central clamp.

13. The system of claim 8, wherein the at least one computer device further configured to base the VECD Model on an elastic-viscoelastic correspondence (E-VC) model of the cylindrical asphalt sample.

14. The system of claim 11, wherein the cylindrical asphalt sample is a core sample taken from an asphalt mixture.

15. A method for testing durability of an asphalt sample, comprising:
placing a cylindrical asphalt sample in a sample-holding apparatus, the cylindrical asphalt sample having a first end and second end thereof, the sample-holding apparatus including a first clamp configured to selectively hold the first end of a cylindrical asphalt sample, and second clamp configured to hold the second end of the cylindrical asphalt sample;
selectively applying a predetermined force against the cylindrical asphalt sample held within the first clamp and second clamp a load cell selectively moveable at a range of predetermined force, the predetermined force applied from load cell configured to apply force on the cylindrical asphalt sample between the first end and second end of the cylindrical asphalt sample via an actuator selectively moving the load cell;
sensing, at the actuator, a resistance of the cylindrical asphalt sample to the predetermined force further;
receiving sensed resistance data from the actuator from the predetermined force applied to the cylindrical asphalt sample;
determining a deflection of the cylindrical asphalt sample based upon one of a Timoshenko Beam Theory (TBT) Model, or a Viscoelastic Continuum Damage (VECD) Model; and
determining a durability measurement of the cylindrical asphalt sample based upon the determined deflection.

16. The method of claim 15, further comprising outputting the durability measurement of the cylindrical asphalt sample.

17. The method of claim 15, wherein sample-holding apparatus further includes a central clamp configured to selectively hold a cylindrical asphalt sample at a point in between the first end and second end of the cylindrical asphalt sample, and the method further includes selectively moving the central clamp.

18. The method of claim 15, further treating the cylindrical asphalt sample as a linear elastic material within a TBT Model.

19. The method of claim 15, further including creating the cylindrical asphalt sample by taking a core sample from an asphalt mixture.

20. The method of claim 15, further including basing the VECD Model on an elastic-viscoelastic correspondence (E-VC) model of the cylindrical asphalt sample.
